(19) United States
(12) Patent Application Publication
(10) Pub. No.: US 2004/0073106 A1
(43) Pub. Date: Apr. 15, 2004

(54) Breast stabilizers having an open lattice structure and imaging methods using same

(75) Inventors: Roberta Lee, Redwood City, CA (US); James W. Vetter, Portola Valley, CA (US); Natalie Hyland, Redwood City, CA (US)

Correspondence Address: YOUNG LAW FIRM, A PROFESSIONAL CORPORATION, 4370 ALPINE ROAD SUITE 106, PORTOLA VALLEY, CA, 94028

(73) Assignee: RUBICOR MEDICAL Inc., Redwood City, CA (US)

(21) Appl. No.: 10/155,704

(22) Filed: May 22, 2002

(51) Int. Cl. A61B 5/05

(52) U.S. Cl. 600/415

(57) ABSTRACT

A breast stabilizer for imaging and invasive medical procedures includes a cage having an open lattice structure. The cage is shaped to conform to at least a portion of a breast and is adapted to removably adhere to the breast by a selective application of suction. A surface area of the breast at least sufficient to allow access for imaging and/or invasive medical procedures is exposed through the open lattice structure of the cage.

### 1. CROSS REFERENCE TO RELATED APPLICATIONS

The present application is related in subject matter to commonly assigned and co-pending U.S. patent application Ser. No. 09/158,215 entitled “Breast Stabilizers and Methods” filed on Sep. 22, 1998 and commonly assigned and co-pending U.S. patent application Ser. No. 09/200,661 entitled “Breast Stabilizers And Imaging And Interventional Methods Using Same” filed on Nov. 25, 1998.

### BACKGROUND OF THE INVENTION

1. Field of the Invention

The present invention pertains to the field of diagnostic and therapeutic medical devices and procedures. More particularly, the present invention relates to the fields of stabilization and imaging of the female breast and related methods.

2. Description of the Related Art

Women aged 40 and over are recommended to undergo an annual screening mammogram to potentially identify a breast cancer in its most early stages of development. By definition, these women are asymptomatic and the lesions within their breasts, if any, are most often non-palpable. Most breast cancers are, therefore, diagnosed by screening mammography. Conventional breast stabilizers and methods are a direct consequence from the technique of mammography. To obtain an acceptable mammographic image, the breast must be compressed and held immobile between two parallel plates. With the use of mammography to localize lesions for diagnostic procedures, the breast had to remain between the two compression plates for imaging and thereafter, to provide a platform from which to conduct the diagnostic procedure. Compression of the breast is mandatory for a stereotactic biopsy, as an adequate mammogram cannot be obtained unless the breast is in compression. During the procedure, the computer calculates the x, y and z coordinates targeting the lesion. The breast stays in compression during the entire localization and biopsy procedure.

Because of the required compression, the placement of the compression plates on the woman's breast determines the skin entry site for the procedure and, therefore, the scar location. Indeed, the position of the breast in the compression device dictates where the incision is to be made. The scar is most always on the side of the breast, whether superior, lateral, inferior or medial. The scar can range from about 5 mm in length to an unsightly 3 cm if a large coring device is used.

From the patient's perspective, such breast compression devices and associated procedures are uncomfortable, awkward and painful. Indeed, such techniques often require the patient to assume an uncomfortable position to fit one of her breasts between the plates, which are then moved toward one another to compress the breast therebetween. This can be quite painful, as the degree of compression necessary to properly stabilize the breast in this manner is quite great. To complicate matters, breast tissue often does not compress evenly, as the breast tissue may have localized regions of relatively greater or lesser densities that may slide against one another, a denser region being likely to push a relatively less dense area out of the way as the breast is compressed between the two parallel plates. In addition, the breast may have been slightly twisted as it was compressed. After an invasive procedure during which the breast was compressed as described above, the breast tissue expands, and the apparent profile of the path followed by the needle or other device (often coring its way through the tissue) may no longer be the straight path taken by the device when the breast was compressed. This results in an often curved or somewhat tortuous cavity in the breast. With a large coring device, this can cause permanent distortion and disfigurement.

Another technique for sampling or excising lesions in the breast involves sonographically targeting the lesion and manually carrying out a fine needle aspiration, core biopsy or vacuum assisted core biopsy. In such a procedure, the breast is not compressed and an ultrasound transducer is typically used to image the breast and the site of interest therein. In ultrasound-guided biopsy, the physician must manually stabilize the breast, hold the ultrasound probe, and perform the biopsy accurately enough to obtain tissue from the lesion. Conventionally, this procedure is carried out by inserting the needle within the breast in an orientation that is as near parallel to the patient's chest wall as possible. The breast stabilization, the operation of the probe, as well as the actual needle biopsy must be carried out simultaneously, all the while maintaining the needle within the focal plane of the ultrasound probe. It is difficult to have an assistant help perform the procedure because if the ultrasound probe and/or needle are not exactly in line and are off by a fraction of a millimeter, then the needle cannot be visualized on the ultrasound monitor. Moreover, any movement of the patient (e.g., coughing, shifting) will also cause the biopsy device and ultrasound probe to misalign.

When the lesion is biopsied with the breast in compression, the cavity left after the biopsy procedure expands as the breast is uncompressed after the procedure. This expanded cavity can cause unsightly disfigurements, particularly when large coring devices are used. It would be advantageous, therefore, to perform the biopsy procedure on an uncompressed breast. However, localization of small breast lesions has conventionally required mammographic imaging. Mammographic imaging, in turn, requires that the breast remain compressed.

Ultrasound imaging is currently used with good results for specific indications, but is generally not used as a screening modality. Indeed, ultrasound is conventionally used to gather additional information about a suspicious area seen on mammography, or about a palpable lesion. Conventionally, it has been difficult to determine conclusively that a suspicious area as seen by ultrasound correlates exactly with that seen during the mammogram. In addition, suspicious microcalcifications seen by mammography are not readily visualized by ultrasound imaging techniques currently available. Therefore, ultrasound conventionally has been of little help in biopsying or excising small, non-palpable cancers or suspicious areas.

In instances where surface ultrasound is effective in localizing a lesion, a manual biopsy procedure may be carried out under surface ultrasonic guidance. In such a directed biopsy procedure, the lesion within the breast is sonographically targeted and a fine needle aspiration, core biopsy or vacuum-assisted core biopsy procedure is carried out. In such a procedure, the breast is not compressed and a surface ultrasound transducer is typically used to image the breast and the site of interest therein. In surface ultrasound-guided biopsy, the physician must manually (i.e., by placing a hand on the breast) stabilize the breast as best as possible, hold the ultrasound probe, and perform the biopsy accurately enough to obtain tissue from the lesion. Conventionally, this procedure is carried out by inserting the needle within the breast in an orientation that is as near parallel to the patient's chest wall as possible. The breast stabilization, the operation of the probe, as well as the actual needle biopsy must be carried out simultaneously, all the while maintaining the needle within the focal plane of the ultrasound probe. It is difficult to have an assistant help perform the procedure because if the ultrasound probe and/or needle are not exactly in line and are off by a fraction of a millimeter, then the needle cannot be visualized on the ultrasound monitor. Moreover, any movement of the patient (e.g., coughing, shifting) will also cause the biopsy device and surface ultrasound probe to misalign.

Other imaging techniques that show promise in imaging small lesions in soft tissue include elastography, sonoelastic imaging and elasticity imaging. Such techniques rely upon differences in tissue structure, stiffness and elasticity to identify certain tissue pathologies. In tissue elastography, for example, an ultrasound of an undisturbed breast and an ultrasound of the same breast under strain (such as under a slight compression) may be digitally compared to generate an elastogram. The elastogram may utilize a certain grayscale range to represent differences in tissue elasticity. Typically, malignant tumors are less elastic than normal healthy tissue, and these tumors may be represented in an elastogram as dark areas that are relatively less elastic than the surrounding healthy tissue. Sonoelastic imaging is similar to elastography but relies upon the imposition of a small mechanical disturbance (such as an externally-applied vibration or mechanical shock) to produce the grayscale representation of the elasticity or stiffness differential of the breast tissue. Such techniques have the potential of identifying small impalpable and previously undetectable lesions. However, elastography and sonoelastic imaging require a stabilized breast, which conventionally required the breast to be in a compressed state, such as is the case when the breast in compressed between the two parallel plates of a mammography device.

Carrying out imaging and/or biopsy procedures on the uncompressed breast would alleviate the disadvantages associated with compressing the breast. Importantly, such procedures on the uncompressed breast would be less painful, would allow more choices for the entry site, would reduce the size of the cavity left after the excisional procedure and would provide a means for excising tissue from the breast in its natural (i.e., uncompressed) state.

### SUMMARY OF THE INVENTION

It is an object of the present invention, therefore, to provide breast stabilizers that allow the breast to the stabilized without compression for interventional and/or imaging procedures. It is another object of the present invention to provide imaging methods using such breast stabilizers.

In accordance with the above-described objects and those that will be mentioned and will become apparent below, a breast stabilizer for imaging and invasive medical procedures includes a first annular member adapted to encircle at least a portion of a base of the breast; a second annular member adapted to encircle at least a portion of an areolar region of the breast, and at least one strut, the strut(s) mechanically coupling the first annular member to the second annular member.

According to further embodiments, the strut(s) include a facing surface that faces the breast when the stabilizer is in use, the aggregate surface area of the facing surface of the strut(s) being preferably less than 50% of the total surface area of the breast. The first annular member may define a first internal lumen and may include a first vacuum port in fluid communication with the first internal lumen. The surface of the first annular member that is adapted to contact the breast may define a plurality of through holes, each of the plurality of through holes being in fluid communication with the first internal lumen. Likewise, the second annular member may define a second internal lumen and may include a second vacuum port in fluid communication with the second internal lumen. The surface of the second annular member that is adapted to contact the breast may define a plurality of through holes, each of the plurality of through holes being in fluid communication with the second internal lumen. The strut(s) may define a third internal lumen and may include a third vacuum port in fluid communication with the third internal lumen. The facing surface of the strut(s) may define a plurality of through holes, each of the plurality of through holes being in fluid communication with the third internal lumen.

Alternatively, the first annular member may define a first internal lumen, the second annular member may define a second internal lumen and the strut(s) may define a third internal lumen, each of the first to third internal lumen being in fluid communication with one another. The stabilizer may further include a suction port in fluid communication with the first to third internal lumen. A suction port may be disposed on one of the strut(s). A suction port may alternatively be disposed on the first and/or the second annular members. Each strut may be shaped to conform to the shape of the breast. According to a further embodiment, three struts mechanically couple the first annular member to the second annular member, each of the three struts being separated from a next adjacent strut by about 120 degrees. Alternatively, four struts may mechanically couple the first annular member to the second annular member, each of the four struts being separated from a next adjacent strut by about 90 degrees. Alternatively still, a single strut or a pair of struts may mechanically couple the fist and second annular members. The first annular member and/or the second annular member may include a substantially rigid outer layer and a relatively softer inner layer, the softer inner layer, in use, being in contact with the breast. The relatively softer inner layer may include, for example, one or more materials selected from the group including polyethylene, polyethylene teraphthalate (PET), PETG, PETE and Nylon.

The present invention is also a breast stabilizer for imaging and invasive medical procedures, comprising a first and a second strut, each including a proximal and a distal end; a third strut defining a first arc configured to allow at least a portion of a nipple-areolar complex of the breast to protrude therethrough, the third strut being attached to the distal ends of the first and second struts, and a fourth strut defining a second arc configured to encircle a portion of a base of the breast, the fourth strut being attached to the proximal ends of the first and second struts.

A first flange may extend from the first strut and a second flange may extend from the second strut, the first and second flanges being configured to secure the stabilizer to a flat surface. The stabilizer may have a truncated generally semi-conical shape adapted to surround that portion of the breast not resting on the flat surface when the stabilizer is in use. The first to fourth struts may each include a substantially rigid outer layer and a relatively softer inner layer, the softer inner layer, in use, being in contact with the breast. The stabilizer may further include a suction port and at least one of the relatively softer inner layers may include a plurality of through holes in fluid communication with the suction port through an interstitial space between the inner and outer layers. The stabilizer may further include a fifth strut that includes a substantially rigid outer layer and a relatively softer inner layer that is in contact with the breast when the stabilizer is in use, the fifth strut mechanically coupling the first strut to the second strut and/or the third strut to the fourth strut. A suction port may be disposed on the fifth strut and the relatively softer inner layer of the fifth strut may define a plurality of through holes in fluid communication with the suction port. At least the third, fourth and fifth struts may be integral to one another and may each include a substantially rigid outer layer and a relatively softer inner layer that defines a plurality of through holes in fluid communication with the suction port. The first flange may define a plurality of through holes, each of the plurality of through holes being in fluid communication with the suction port, thereby enabling the stabilizer to be secured by suction to the flat surface when suction is applied to the suction port. An adhesive layer may be disposed on the surface of the first and second flanges that faces the flat surface when the stabilizer is in use. The relatively softer layer may include, for example, one or more materials selected from the group including polyethylene, polyethylene teraphthalate (PET), PETG, PETE and Nylon. The first and second arcs may have a generally semicircular shape. The stabilizer may further include a third flange extending from the fourth strut, the third flange being oriented to face the woman's chest wall when the stabilizer is in use. The third flange may also define a plurality of through holes in fluid communication with the suction port. The suction port may include a (elastomeric, for example) valve adapted to maintain a pressure differential between ambient pressure and the pressure in the interstitial space between the inner and outer layers. The fifth strut may be attached only to the third and fourth struts. Alternatively, the fifth strut may be attached only to the first and second struts. The stabilizer may have a shape that approximates the shape of an exposed portion of a breast as the breast rests on a substantially flat surface.

The present invention is also a method of imaging a female breast, comprising a step of stabilizing the breast by placing a breast stabilizer thereon, the stabilizer including a first annular member adapted to encircle at least a portion of a base of the breast; a second annular member adapted to encircle at least a portion of a peri-areolar region of the breast, and at least one strut, the at least one strut mechanically coupling the first annular member to the second annular member, and a step of imaging the breast through an exposed portion of the stabilized breast.

The imaging step may be carried out using an ultrasound-imaging device and/or a magnetic resonance imaging (MRI) device, for example. The imaging step may be carried out using an intra-tissue ultrasound device inserted adjacent the per-areolar region of the stabilized breast. The imaging step may include a step of simultaneously disposing two external ultrasound-imaging devices against exposed areas of the stabilized breast. The imaging step may be carried out through an elastographic tissue imaging technique. The elastographic imaging step may include the steps of carrying out an imaging step on the stabilized breast in an undisturbed state; subjecting the stabilized breast to a mechanical strain; imaging the strained breast, and comparing images obtained from the breast in the undisturbed state and strained breast. The stabilizer, in use, may attach to the breast using suction and the force applied to the breast by the suction may be varied to generate the mechanical strain. The stabilizer may include a suction port and the first and second annular members and the at least one strut may include a substantially rigid outer layer and a relatively softer inner layer facing the breast when the stabilizer is in use, the inner layer defining a plurality of through holes in fluid communication with the suction port through an interstitial space between the inner and outer layers. The subjecting step may then include a step of varying a differential between ambient pressure and a pressure within the interstitial space.

According to another embodiment thereof, the present invention includes a method of imaging a female breast, comprising the steps of stabilizing the breast by placing a breast stabilizer thereon, the stabilizer including a first and a second strut, each of the first and second struts including a proximal and a distal end; a third strut defining a first arc configured to allow at least a portion of a nipple-areolar complex of the breast to protrude therethrough, the third strut being attached to the distal ends of the first and second struts and a fourth strut defining a second arc configured to encircle a portion of a base of the breast, the fourth strut being attached to the proximal ends of the first and second struts, and imaging the breast through an exposed portion of the stabilized breast.

The imaging step may be carried out using an external ultrasound imaging device and/or an MRI device. The imaging step may be carried out using an intra-tissue ultrasound device inserted adjacent the per-areolar region of the stabilized breast. The imaging step may include a step of simultaneously disposing two external ultrasound-imaging devices against the stabilized breast. The imaging step may be carried out through an elastographic tissue imaging technique. Such an elastographic imaging step may include steps of carrying out an imaging step on the stabilized breast in an undisturbed state; subjecting the stabilized breast to a mechanical strain; imaging the strained breast, and comparing images obtained from the breast in the undisturbed state and strained breast. The stabilizer, in use, may attach to the breast using suction and the force applied to the breast by the suction may be varied to generate the mechanical strain. The stabilizer may include a suction port and at least one of the first to fourth struts may include a substantially rigid outer layer and a relatively softer inner layer, the inner layer defining a plurality of through holes in fluid communication with the suction port through an interstitial space between the outer and inner layers. The subjecting step may include a step of varying a differential between ambient pressure and a pressure within the interstitial space.

The present invention may also be viewed as a breast stabilizer for imaging and invasive medical procedures, comprising a cage having an open lattice structure, the cage being shaped to conform to at least a portion of a breast, the cage being adapted to removably adhere to the breast by a selective application of suction, wherein a surface area of the breast at least sufficient to allow access for imaging and/or invasive medical procedures is exposed through the open lattice structure of the cage. The stabilizer may be re-usable or the stabilizer may be a one-time use and disposable device. The open lattice structure of the cage may be configured to expose, for example, at least 40% of the surface area of the breast. The cage may be configured to expose at least a portion of a peri-areolar region of the breast when in use.

### BRIEF DESCRIPTION OF THE DRAWINGS

FIG. 1 shows a perspective view of a breast stabilizer for imaging and invasive medical procedures, according to an embodiment of the present invention.

FIG. 2 shows a top view of the breast stabilizer of FIG. 1.

FIG. 3 shows a view of the underside of the breast stabilizer of FIG. 1, showing the surface thereof that faces and contacts the patient's breast when the stabilizer is in use.

FIG. 4 shows a cross-sectional view of the stabilizer of FIG. 1, taken along lines AA′.

FIG. 5 shows a perspective view of a breast stabilizer for imaging and invasive medical procedures, according to another embodiment of the present invention, wherein the three struts are disposed about 120 degrees from one another.

FIG. 6 shows a perspective view of a breast stabilizer according to another embodiment of the present invention, wherein the half breast stabilizer is in use on a patient's breast.

FIG. 7 shows another embodiment of a breast stabilizer for imaging and invasive medical procedures, according to the present invention

FIG. 8 shows still another embodiment of a breast stabilizer, according to the present invention.

FIG. 9 shows a bottom view of the half breast stabilizer of FIG. 6, showing the surface thereof that faces and contacts the patient's breast when the breast stabilizer is in use.

FIG. 10 shows a cross-sectional view of a vacuum port suitable for use with the breast stabilizers of FIGS. 1-9.

FIG. 11 is a flowchart of a method of biopsying and/or excising a breast lesion using a stabilizer according to an embodiment of the present invention

### DETAILED DESCRIPTION

1. Structural and Functional Description

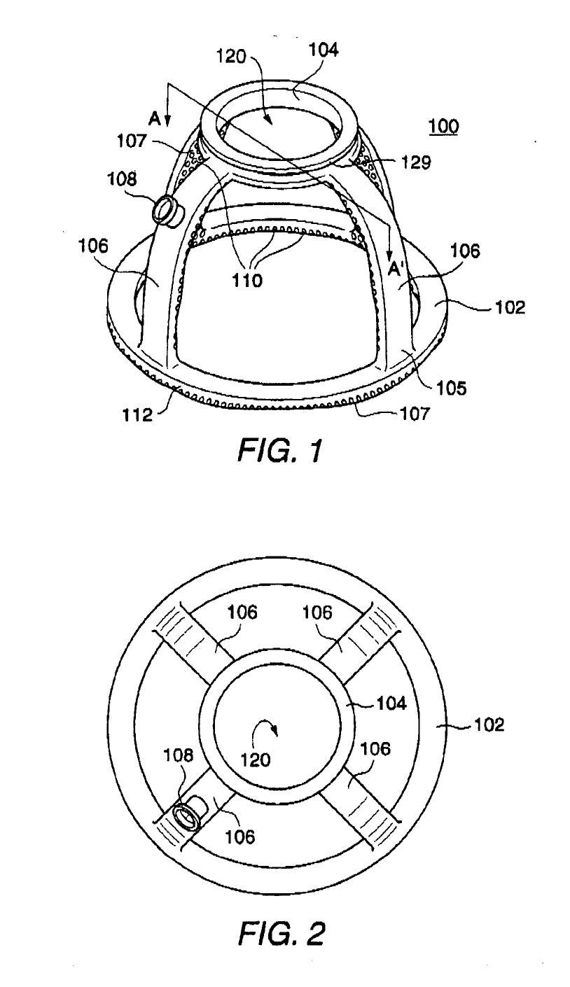

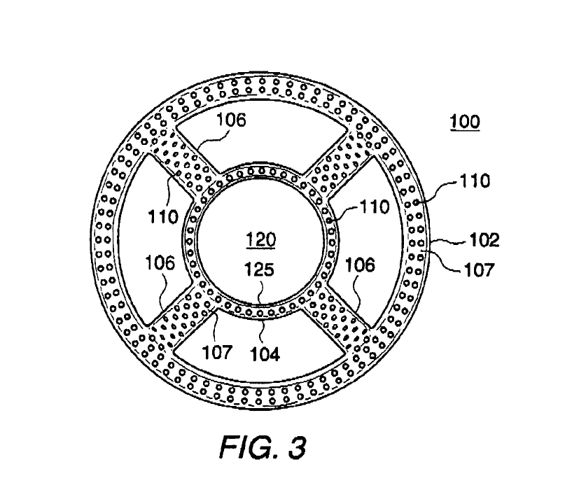

FIG. 1 shows a perspective view of a breast stabilizer 100 for imaging and invasive medical procedures, according to an embodiment of the present invention, whereas FIG. 2 shows a top view thereof and FIG. 3 shows the underside of the breast stabilizer of FIG. 1, showing the surface thereof that faces and contacts the patient's breast when the stabilizer is in use. As shown in FIGS. 1-3, the stabilizer 100 includes a first annular member 102 adapted to encircle at least a portion of the base of the patient's breast (not shown in FIG. 1) and rest against the patient's rib cage. The stabilizer 100 also includes a second annular member 104 that is adapted to encircle at least a portion of the areolar region of the breast when the stabilizer 100 is in use. At least one strut 106 mechanically couples the first annular member 102 to the second annular member 104. The first and second annular members 102, 104 are maintained separated from one another by the strut(s) 106. Together, the first and second annular members 102, 104 and the strut(s) 106 form an at least semi-rigid assembly that generally approximates the shape of the female breast in its natural, uncompressed state. The annular members 102, 104 and/or the strut(s) 106 may be formed of a rigid or semi-rigid material, including plastic and/or metal, for example. Functionally, the assembly 102, 104 and 106, when fitted on the patient's breast, imparts the patient's breast with a higher degree of stability than would otherwise be the case if the stabilizer 100 were not used. The second annular member 104 may also define a lip 129 onto which interventional and/or imaging devices may be clamped or otherwise secured.

To fit the stabilizer 100 onto a patient, the patient may lie prone on her back, side or front and the stabilizer 100 may be fitted onto her breast such that the first annular member 102 rests against the patient's rib cage and encircle the base of her breast and such that the nipple and at least a portion of the nipple-areolar complex protrudes from the (substantially circular) opening 120 of the second annular member 104.

According to an embodiment of the present invention, the strut(s) 106 (four such struts 106 being shown in the embodiment of FIG. 1) define a facing surface 114 that faces the breast when the stabilizer 100 is in use. The aggregate surface area of the facing surface 114 is preferably less than about 50% of a total surface of the breast. Preferably, the facing surface area 114 constitutes less than about 40% of the total surface area of the breast. For example, the aggregate area of the facing surface 114 of the strut(s) 106 is 25% or less of the total surface area of the breast, to allow ready access to the breast surface by imaging and/or invasive surgical instruments, as will be further detailed below.

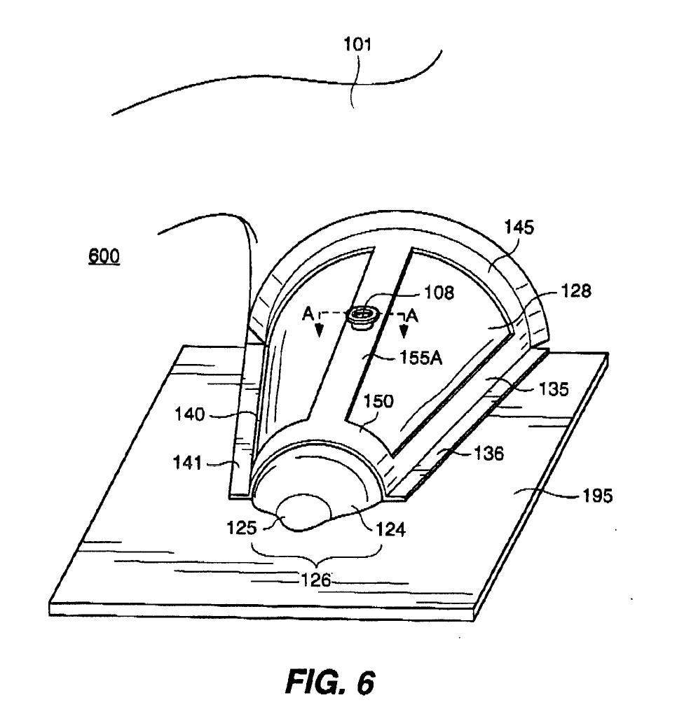

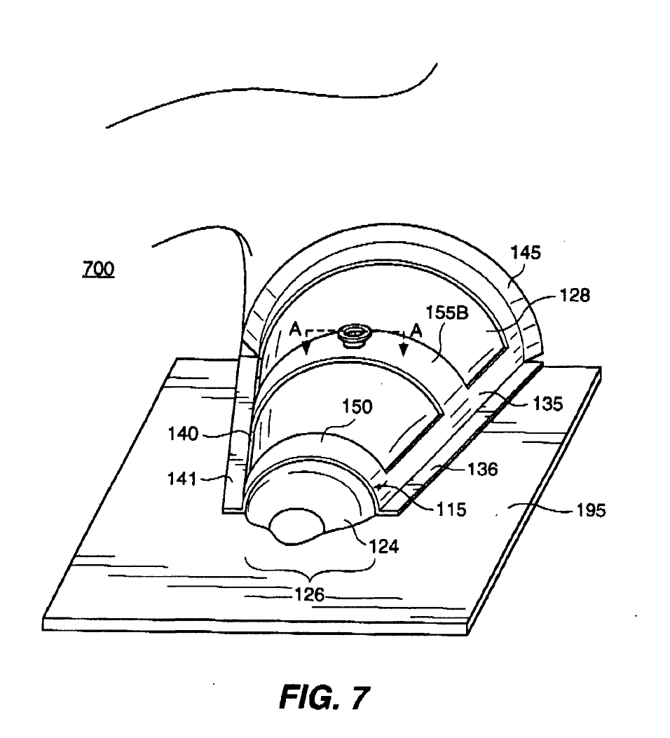

Considering now FIGS. 1-4 collectively, each of the struts(s) 106 and each of the first and second annular members 102, 104 may be formed of an inner layer 107 joined to an outer layer 105. The inner layer 107 is configured to conform closely to the shape of the breast with which it comes into contact with when the stabilizer 100 is in use. As the inner layer 107 is preferably relatively softer and more flexible than the outer layer 105, it is able to conform closely to the shape of the underlying breast. The inner layer may include one or more materials such as polyethylene, polyethylene teraphthalate (PET), PETG, PETE and Nylon, for example. The relatively softer inner layer 107 is well adapted to conform or to mold itself to the shape of the breast as the stabilizer 100 is pressed thereon. The outer layer 105 may be substantially rigid, so as to allow the physician to apply force thereon without collapsing the stabilizer 100. An opening 120 is disposed within the outer and inner layers 105, 107, the opening 120 being aligned with the nipple/areolar complex (shown at reference numeral 126 in FIGS. 6-8) of the breast. A rigid rim 125 may surround the periphery of the opening 120 and may rise above the surface of the outer layer 105. Alternatively, the rigid rim 125 may be omitted altogether. The opening 120 and the rim 125 are preferably centered on the nipple/areolar complex of the breast and preferably expose at least a portion of the areola surrounding the nipple when the stabilizer 100 is in use. More preferably, the diameter of the opening 120 and the diameter of the rim 125 are such as to expose and to closely surround the circumference of the areola. The height of the rim 125 above the surface of the outer layer 105 and the inner diameter of the rim 125 should preferably be such as to allow unfettered access by surgical needles and other instruments to the entire breast. The rim 125 may includes a lip 129 or other similar structure to allow one or more instruments to be secured (e.g. clamped) thereto. The lip 129 may be integral to the rim 125, and the rim 125 may be integrally formed with the outer layer 105. Alternatively, the rim 125 may be detachable, and fitted to the stabilizer 100 by friction (for example). In this manner, an assortment of rims 125 having different lip configurations or heights may be attached to the device 100 to allow a variety of medical instruments to be secured thereto. Preferably, the rim 125, when present and installed, is sealed to the inner layer 107.

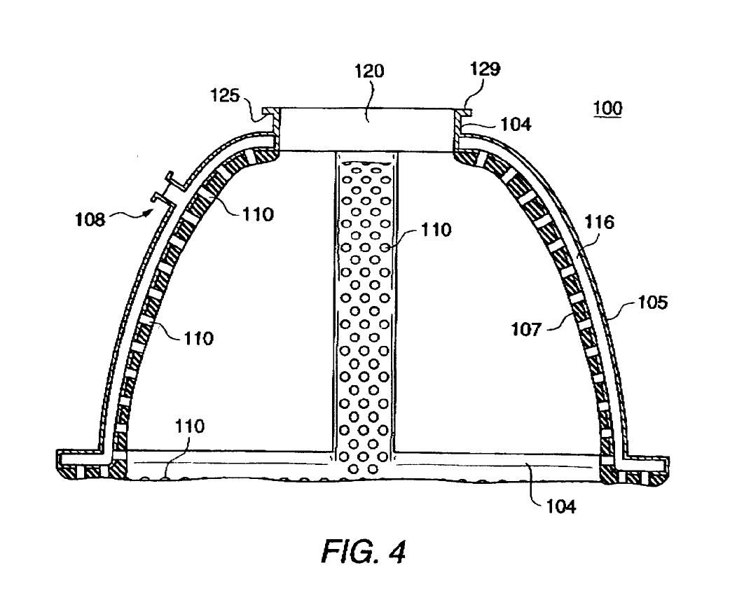

As shown most clearly in FIG. 4, the strut(s) 106 of the stabilizer 100 may define an interstitial space 116 (or an internal lumen) between the inner layer 107 and the outer layer 105. The stabilizer 100 may also include a vacuum port 108 in fluid communication with (i.e., open to) the interstitial space 116. A plurality of through holes 110 may be defined through the inner layer 107. Each of the plurality of through holes 110 may be in fluid communication with (i.e., open to) the interstitial space 116. The vacuum port 108 may be mounted to the outer layer 105 of one of the struts 106, for example. According to the present invention, when the stabilizer 100 is placed over the patient's breast, a vacuum may be applied to (e.g., air may be drawn through) the vacuum port 108 (by means of a vacuum pump for example). The vacuum then tends to draw the breast, already in intimate contact with the inner layer 107, closer to the stabilizer 100 and tends to seal the breast thereto, as the through holes 110 are open to the interstitial space 116 through which the vacuum is being drawn. In sealing the breast to the inner layer 107 of the stabilizer 100, the breast may expand somewhat. The number and uniform spacing of the through holes 110 insures that the breast is drawn toward the strut(s) 106 with a uniformly distributed force, increasing the patient's comfort as suction is applied through the vacuum port 108.

Similarly, the first annular member 102 may include an outer layer and an inner layer that faces and contacts the patient's breast when the stabilizer 100 is in use. The inner and outer layer of the first annular member 102 may also define an interstitial space (or lumen) therebetween. A plurality of through holes 110 may also be defined within the inner layer of the first annular member 102. Each of the plurality of through holes 110 is in fluid communication with the interstitial space defined between the inner and outer layers of the first annular member 102. A vacuum port may also be fitted to the first annular member 102, the vacuum port enabling a vacuum to be drawn through the port and the interstitial space of the first annular member 102. As before, the plurality of through holes 110, in concert with the vacuum port and the interstitial space of the first annular member, serve to draw the stabilizer 100 closer to the patient's breast and to seal the stabilizer 100 thereto.

In like manner, the second annular member 104 may include an outer layer and an inner layer that faces the patient's breast when the stabilizer 100 is in use. The inner and outer layer of the second annular member 104 may also define an interstitial space (or lumen) therebetween. A plurality of through holes 110 may also be defined within the inner layer of the second annular member 104. Each of the plurality of through holes 110 is in fluid communication with the interstitial space defined between the inner and outer layers of the second annular member 104. A vacuum port may also be fitted to the second annular member 104, the vacuum port enabling a vacuum to be drawn through the port and the interstitial space of the second annular member 104. The plurality of through holes 110, in concert with the vacuum port and the interstitial space of the second annular member 104, serve to draw the stabilizer 100 closer to the patient's breast and to seal the stabilizer 100 thereto.

As each of the first and second annular members 102, 104 and the strut(s) 106 may include independent interstitial spaces (or lumens) and vacuum ports, the suction applied to each of these members and struts may be varied independently by the selective application of suction therein. Alternatively, the first annular member 102 may define a first interstitial space (or lumen), the second annular member 104 may define a second interstitial space (or lumen) and each of the struts 106 may define a third interstitial space (or lumen), each of the first to third interstitial spaces (such as shown at 116 in FIG. 4) being in fluid communication with one another (i.e., open to one another). In this embodiment, the stabilizer 100 may further include a single suction port 108 in fluid communication with the first to third interstitial spaces. According to this embodiment, suction may be applied uniformly to the breast through the single suction port 108, the breast being drawn to the inner layer 107 of the stabilizer under the influence of the suction applied to the breast through the through holes 110 defined in the inner layer 107.

The suction port is shown in FIG. 4 as being disposed on one of the struts 106. However, the suction port 108, according to other embodiments of the present invention, may alternatively be disposed on the first or second annular members 102, 104 and/or on one or more of the struts 106. Disposing more than one suction port 108 on the stabilizer may provide ready access thereto by the physician, irrespective of the woman's body position during the imaging and/or interventional procedure in which the stabilizer 100 is utilized.

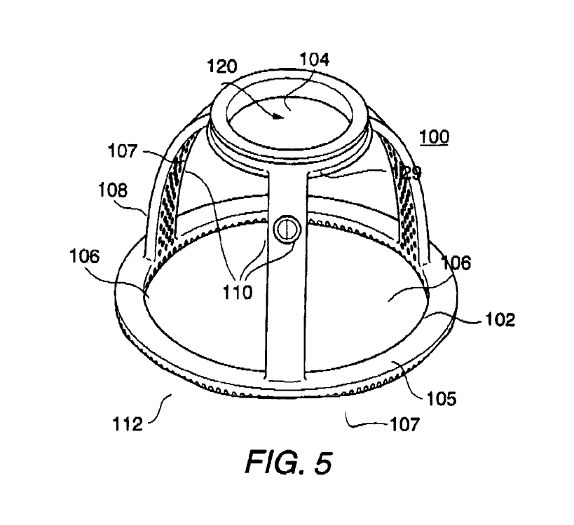

As shown in FIGS. 1-4, the stabilizer 100 includes four discrete struts 106, each mechanically coupling the first annular member 102 to the second annular member 104. According to an embodiment of the present invention, each of the four struts 106 may be separated from the next adjacent strut 106 by about 90 degrees, although other radial strut distributions are possible. Indeed, FIG. 5 shows an embodiment of the stabilizer 100 that includes three struts 106 mechanically coupling the first annular member 102 to the second annular member 104, each of the three struts 106 being separated from the next adjacent strut 106 by about 120 degrees, although other strut distributions are possible. Alternatively, only one or two struts 106 may be provided, depending upon the application. A greater number of struts 106 may also be provided, albeit at the expense of reducing the exposed surface area of the woman's breast accessible to the physician when the device is in use securing and stabilizing the breast.

In use, the breast stabilizer 100 is placed over the breast (not shown), and the opening 120 thereof is aligned and centered with the nipple/areolar complex thereof. Once the stabilizer 100 is disposed over the woman's breast, the air present in the interstitial space(s) 116 (or lumens) defined between the outer and inner layers 105, 107 is drawn through the suction port(s) 108, creating a partial vacuum (lower air pressure than ambient) within the interstitial space(s) 116. The partial vacuum in the interstitial space (s) 116 tends to draw some of the air that still is present between the breast and the inner layer member 107 in through the through holes 110 and the suction port 108. In turn, the breast is drawn towards and in intimate contact with the facing surface 114 of the inner layer 107, thereby sealing the stabilizer 100 to the breast, preferably leaving the nipple and at least a portion of the areola exposed through the opening 120 thereof. The opening 120 may thereafter become the physician's primary access port to the breast for invasive procedures. The suction through the suction port 108 may be accomplished by means of a syringe and plunger arrangement attached to the suction port 108, or through other conventional means known to those of skill in this art. As the inner layer 107 is relatively more flexible and softer than the outer layer 105, and as the air between the breast and the inner layer 107 is drawn out through the through holes 110 and the suction port 108, the inner layer 107 tends to mold itself to the breast, thereby securely adhering the stabilizer 100 to the patient's breast. When it is desired to release the patient's breast from the stabilizer 100, low-pressure air may be directed into the device through the suction port or ports 108. In this manner, the air drawn into (and/or forced through) the port(s) 145 will gently break the seal created by the previously applied suction, thereby allowing the stabilizer 100 to be removed from the patient.

When suction is employed to secure the stabilizer 100 to the breast, the breast may expand somewhat within the breast stabilizer 100. This slight expansion of the breast may actually aid in the visualization thereof by, for example, ultrasound and/or sonoelastic or sonographic techniques. By carefully matching the size and shape of the breast stabilizer 100 to the patient's breast size and shape, however, a close fit may be achieved. This close fit will limit the degree of expansion (if any) of the breast as suction is applied through the suction port(s) 108 of the stabilizer 100.

FIGS. 6 through 8 show other embodiments of the present invention. Considering now FIGS. 6-8 collectively, the breast stabilizers 600, 700 and 800 are depicted in use, and secured to a flat surface 195. The flat surface 195, however, is shown for ease of description only and forms no part of the present invention. For example, the flat surface 195 may be a lower compression plate of a mammography-imaging device (not shown). The breast stabilizers 600, 700 and 800 have a shape that conforms generally to the superior and lateral sides of a female breast 128. This shape may, in general terms, be characterized as a truncated semi-conical shape, although the stabilizer's size and shape, according to the present invention, may be adapted to fit various breast sizes and shapes.

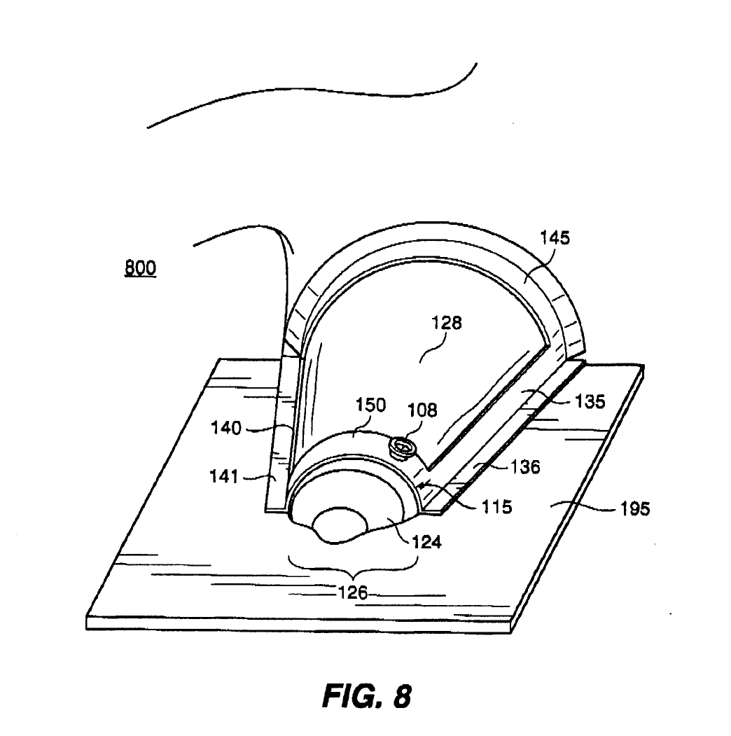

The breast stabilizers 600, 700, 800, according to the present invention, generally conforms to the size and shape of a female breast 128 as the breast 128 rests on a flat surface 195, such as a lower compression plate 195 of a mammography machine. Therefore, most of the inferior portion of the breast 128 shown in FIGS. 6-8 lies substantially flat against the surface 195. For purposes of the present invention, the superior portion of the breast 128 may be thought of that portion of the breast 128 that is above a plane through the nipple and perpendicular to the chest wall and the inferior portion of the breast may be thought of that portion of the breast 128 that lies below that plane, when the woman is in an upright position. Alternatively, the superior portion of the breast may be thought of as that portion of the breast that does not rest on the flat surface 195, irrespective of the position of the woman. As shown in FIGS. 6-8, each of the breast stabilizers 600, 700, 800 includes a first strut 135 and a second strut 140. Each of the first and second struts 135, 140 includes a proximal end adjacent the patient's chest and a distal end adjacent the nipple 125 and areola 124 (together constituting the nipple-areolar complex 126). A third strut 150 defines a first arc configured to encircle at least a portion of a nipple-areolar complex 126 of the breast 128, thereby allowing at least a portion of the complex 126 to protrude through the first arc formed by the third strut 150. The third strut 150 may be attached to the respective distal ends of the first and second struts 140, 135. A fourth strut 145 defines a second arc configured to encircle at least a portion of a base of the breast 128, the fourth strut 145 being attached to the respective proximal ends of the first and second struts 135, 140. As shown in FIGS. 6-8, a first flange 136 may extend from the first strut 135 and a second flange 141 may extend from the second strut 140, the first and second flanges 136, 141 being configured to secure the stabilizer to a flat surface, such as surface or plate 195. The flanges 136, 141 may be releasably attached to the flat surface 195 by means of an adhesive, a mechanical fastener (not shown) or by means of suction. As shown in FIG. 6, the stabilizer 600 may include a fifth strut 155A that mechanically couples the third strut 150 to the fourth strut 145. The fifth strut may alternatively mechanically couple the first strut 135 to the second strut 140, as shown at 155B in FIG. 7. Alternatively still, the fifth strut may be omitted altogether, as shown at FIG. 8, thereby affording the physician access to the greatest exposed surface area of the breast 128 possible.

As with the stabilizer 100 of FIG. 1, the first to fifth struts 135, 140, 150, 145, 155A, 155B of the stabilizers 600, 700, 800 of FIGS. 6-8 may each include a substantially rigid layer and a relatively softer inner layer that is in contact with the breast 128 when the stabilizer 600, 700, or 800 is in use. The inner and outer layers may be separated from one another and together define an interstitial space similar to that shown at 116 in FIG. 4. A suction port 108 may be disposed on one or more of the first to fifth struts 135, 140, 150, 145, 155A, 155B. For example, the suction port 108 is disposed on the fifth strut 155A in FIG. 6, the fifth strut 155B in FIG. 7 and on the third strut 150 in FIG. 8. The suction port 108, wherever disposed, is in fluid communication with the interstitial space (or lumen(s)) defined between the substantially rigid outer layer and the relatively softer inner layer.

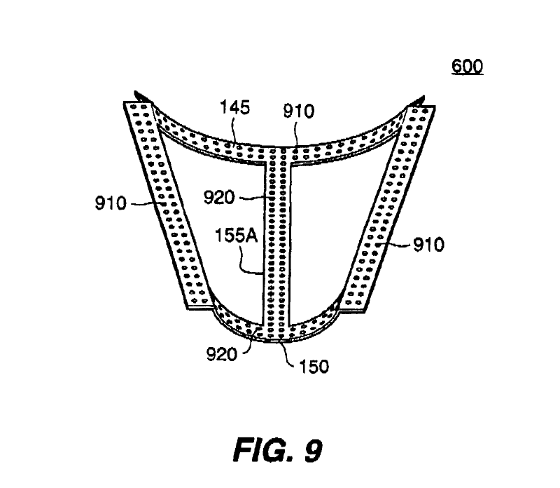

FIG. 9 is a bottom view of the half breast stabilizer 600 of FIG. 6, showing the surface (referenced at 920) of the inner layer thereof, the surface 920 facing and contacting the patient's breast 128 when the breast stabilizer 600 is in use. As shown in FIG. 9, the relatively softer inner layer of the stabilizer 600 defines a plurality of through holes 910, each of the through holes 910 being in fluid communication with the interstitial space (described above) defined between the stabilizer's substantially rigid outer layer and relatively softer inner layer. By applying suction to the suction port 108 (not visible in FIG. 9) after the stabilizer 600 is placed on the breast 128, the physician may cause air to be drawn through the through holes 910, the interstitial space and the suction port 108, the stabilizer 600 will securely adhere to the breast 128 and to the flat surface 195 (FIGS. 6-8), thereby stabilizing the breast 128 for later surgical and/or imaging procedures.

The stabilizers 600, 700, 800 may also be described as forming an open lattice structure wherein the lattice is formed by the first to fourth struts, such as shown at 135, 140, 150, 145 and optionally by the fifth struts 155A and 155B. Together, the struts form a truncated and generally semi-conical shape that is adapted to surround that portion of the breast 128 not resting the flat surface 195 when the stabilizer 600, 700, 800 is in use. Described differently, the stabilizer 600, 700, 800 may have a shape that approximates the shape of an exposed portion of the breast 128 as the breast 128 rests on a substantially flat surface, such as shown at 195. According to an embodiment of the present invention, the relatively softer layer (107 in FIG. 4) of the stabilizers 100, 600, 700 and 800 may include one or more materials selected from a group including polyethylene, polyethylene teraphthalate (PET), PETG, PETE and Nylon, for example. Functionally, the relatively softer layer 107 may be formed of and/or include any surgically appropriate soft material that is able to mold itself to the patient's breast 128 while enabling air to be drawn through the through holes 110, 910 to secure the stabilizer to the patient's breast 128.

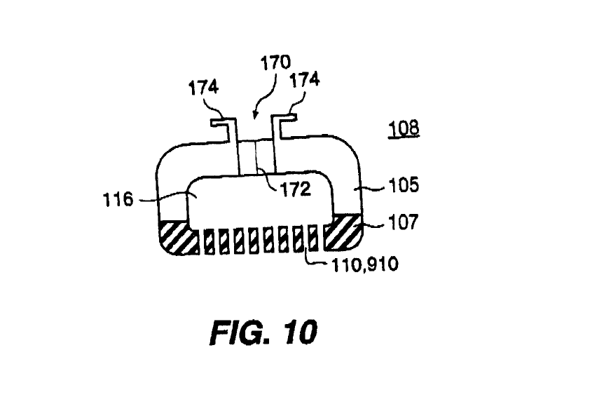

FIG. 10 shows a cross-sectional view of the suction port 108, taken along lines AA′ (FIG. 6). As shown, the suction port 108 may include a valve 170 adapted to maintain a pressure differential between the ambient atmospheric pressure and the pressure within the interstitial space 116 between the inner and outer layers 107, 105. The valve 170 may be an elastomeric valve and include a plug of elastomeric material that includes a slit 172 through which a syringe, for example, may be introduced to draw air from within the interstitial space 116 to create a partial vacuum therein to secure the stabilizer 100, 600, 700 or 800 to the breast 128. The suction port 108, as shown in FIG. 10, may be integrally formed with the inner and outer layers 107, 106 of the stabilizer 100, 600, 700 or 800. Alternatively, the suction port 108 may be an element of the stabilizer that is separate and distinct from therefrom, and fitted thereto by means of friction, adhesive or any other means known to those of skill in this art. By inserting a syringe (not shown) or other thin conduit through the slit 172 of the elastomeric valve 170 and retracting the plunger of the syringe, a partial vacuum may be drawn within the interstitial space 116 between the outer layer 105 and the inner layer 107 to secure the stabilizer 100, 600, 700, 800 to the breast 128. To release the stabilizer 100, 600, 700, 800 from the breast 128, air may allowed within the interstitial space 116 through the elastomeric valve 170, which should reduce the differential pressure between ambient and that present in the interstitial space 116.

The stabilizer 100, 600, 700, 800 is preferably sterile, and may either be disposable or made from materials that are suitable for multiple uses and that may be autoclaved. Disposable stabilizers, however, minimize the risk of transmitting harmful viruses, such as the HIV or hepatitis B virus, or other diseases communicable by bodily fluids.

2. Interventional and Imaging Methodology

The lip 129 of the second annular member 104 may serve as a platform on which to clamp imaging and/or other instruments, such as excisional and ultrasound devices and the like. The large surface area of the stabilized breast 128 that is exposed between the strut(s) 106, 135, 140, 150, 145, 155A, 155B allows great flexibility in the use and emplacement of surface ultrasound devices to ultrasonically image the internal structure of the breast 128. According to the present invention, the physician may locate the target area within the breast 128 using an imaging device, such as an ultrasound probe. In that case, an acoustic coupling medium (such as a commonly available ultrasound gel) may be applied to the exposed portion of the breast. For best results, the ultrasound device should be set at a frequency that balances the degree of penetration of the acoustic energy into the breast tissue with the desired or necessary resolution. Preferably, the ultrasound device should be tuned between a range selected from about 7.5 MHz to about 15 MHz. For example, the ultrasound device may be tuned at a frequency of about 10 MHz.

Once the site of interest is located, with the ultrasound device(s) may be clamped to a stable support to free the surgeon's hands during the actual excisional or biopsy procedure. By stabilizing the breast 128 with a stabilizer as shown at 100, 600, 700 or 800, the physician need no longer insert the biopsy needle parallel to the patient's chest wall, as previously necessary using conventional sonographically guided techniques. Indeed, as the breast 128 is well stabilized within the breast stabilizer 100, 600, 700 or 800 and correctly imaged by the imaging device(s), there is no longer any need to insert the needle parallel to the chest wall. By inserting the needle or excisional device through the opening 120, the needle or excisional device is advantageously oriented substantially parallel to the lactiferous duct structures within the breast 128. Scars along the border of the areola are much less noticeable than scars of similar length made in the side surface of the breast. The areola is, therefore, an ideal point of entry into the breast, as compared with the side top or bottom of the breast. The present inventions, therefore, stabilize the breast 128 while providing an ideal access port (opening 120) that is aligned (when the stabilizer is in use) with the nipple/areolar complex 126. Likewise, with reference to FIGS. 6-8, the primary breast access port when the stabilizer 600, 700 or 800 is used is formed by the a third strut 150 that defines an arc configured to allow at least a portion of the nipple-areolar complex 126 of the breast 128 to protrude therethrough. By stabilizing the breast 128 as disclosed herein, the physician is provided with free access to a stabilized and uncompressed breast 128 in a direction that is substantially aligned with the breast's lactiferous ducts. If needed, the physician may also insert imaging and/or interventional devices through any exposed surface area of the stabilized breast 128.

The present inventions are also well adapted to allow true 3-D real time (or near real time) ultrasonic imaging of a stabilized and uncompressed breast 128. Indeed, by deploying two or more imaging devices on the exposed portions of the stabilized breast 128 (the imaging devices being separated by, for example, about 15 degrees or more), a three dimensional representation of the inner breast structure may be rendered on a computer display terminal for the physician's reference during the procedure itself. As the tip of the biopsy needle or other interventional tool inserted in the breast 128 (through the exposed peri-areolar region thereof, for example) may be opaque to ultrasounds, the surgeon may guide the instrument within the breast while simultaneously consulting a real time or near real time digital representation of the breast 128. In this manner, a feedback loop between the surgeon and the digital representation of the uncompressed and stabilized breast 128 is now possible. The surgeon may then confirm, for example, that the lesion site has indeed been reached or that the entire lesion has been excised by consulting the digital representation of the breast 128 during the procedure.

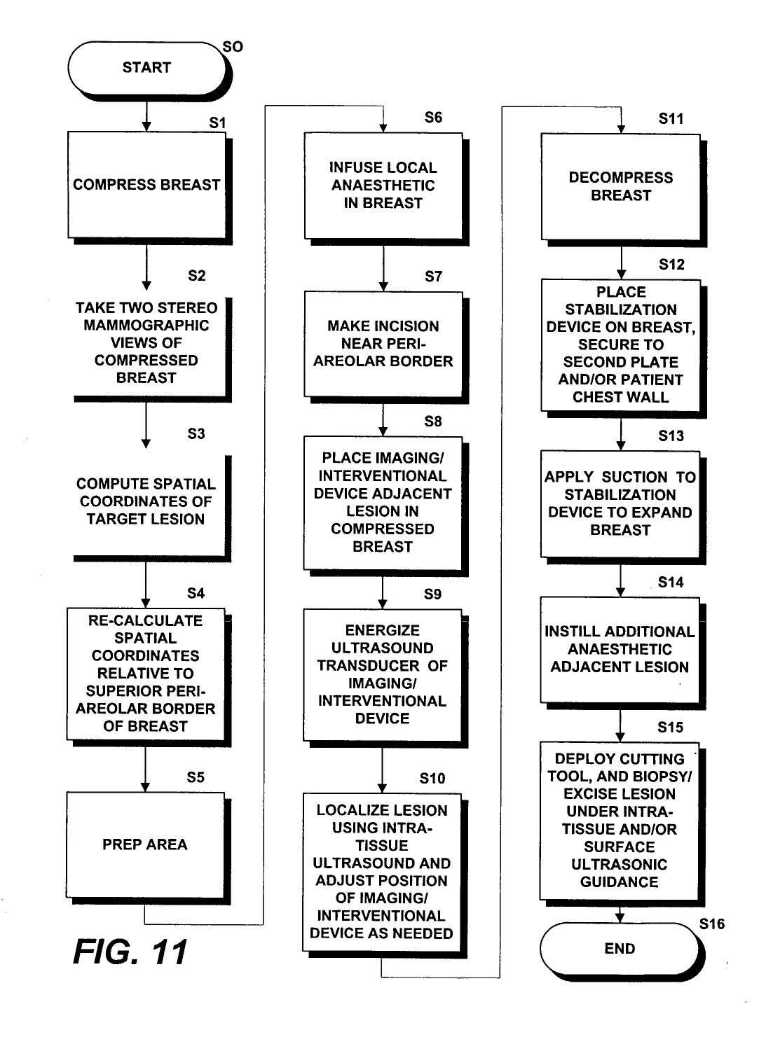

FIG. 11 is a flowchart of a method of biopsying and/or excising a breast lesion using a stabilizer 100, 600, 700 or 800 according to an embodiment of the present invention. The method begins at step S0. At step S1, the breast may be compressed between a first plate and a second plate. For example, the first plate may include an upper compression plate of a mammography device and the second plate may include a lower compression plate thereof. In step S2, at least two stereo mammography views may be taken of the compressed breast. Step S3 calls for the computation, from the stereo views, of the spatial coordinates (for example, x, y, z rectangular coordinates) of the target lesion within the breast 128. Steps S2 and S3 are optional, as indicated by the dashed lines, it being possible to use standard mammography localization techniques. In step S4, the spatial coordinates computed in S3 are re-calculated, so that the coordinates indicate the position of the lesion within the breast 128 relative to the (e.g., superior) border of the peri-areolar complex 126 of the breast 128. In step S5, the area (e.g., the nipple-areolar complex 126) may be surgically prepped with, for example, Betadine. Local anesthetic is infused in the breast 128 in step S6 and an incision is made at or near the peri-areolar border.

In step S8, an imaging and/or interventional device (such as disclosed, for example, in commonly assigned U.S. patent application Ser. No. 09/417,520 filed on Oct. 13, 1999 and entitled “Excisional Biopsy Device and Methods” and/or U.S. patent application Ser. No. 09/565,611 filed on May 4, 2000 and entitled “Excisional Biopsy Devices and Methods” the disclosures of each being hereby incorporated herein) may be inserted through the incision made in step S7 and the device may be advanced through the breast 128 to a position adjacent the target lesion in the compressed breast. Step S8 is preferably carried out under stereotactic guidance to the recalculated spatial coordinates obtained in step S4. The position of the imaging/interventional device may be confirmed using mammography. At step S9, the ultrasound transducer of the imaging/interventional device is energized. Using at least such intra-tissue ultrasound, the lesion may be identified and localized and the imaging/interventional device precisely positioned relative to the lesion within the compressed breast.

Steps S11 through S16 may be carried out on an uncompressed breast. In step S11, the breast may be decompressed. For example, the upper compression plate of the mammography device may be moved and/or removed, thus allowing the breast to decompress. In step S12, the breast stabilizer 100, 600, 700 or 800 may be fitted over the breast 128, while the breast 128 rests on the second plate (only in the case of stabilizers 600, 700 or 800, the stabilizer 100 needing no such plate 195), such as the lower compression plate of the mammography device or another flat surface, such as shown at 195 in FIGS. 6-8. The stabilizer 600, 700 or 800 may then be secured to the second plate 195 (embodiments of FIGS. 6-8) and/or to the patient's chest wall (embodiments of FIGS. 1 and 5). It is preferable that the patient remains substantially immobile during and after step S11 as the breast is decompressed. In step S13, suction may be applied to the stabilizer 100, 600, 700 or 800 according to the present invention through, for example, the suction port 108. This causes fluid (air, for example) to be drawn through the plurality of through holes 110, 910 through the interstitial space(s) 116 between the outer layer 105 and the inner layer 107 and through the suction port 160. The force of the suction draws the breast 128 in intimate contact with the inner layer 107 of the stabilizer 100, 600, 700 or 800, slightly expanding the breast volume and stabilizing the breast 128 within the stabilizer 100, 600, 700 or 800.

In step S14, additional anesthetic may be infused within the breast 128 as needed. Finally, in step S15, an excisional tool may be deployed and the lesion biopsied and/or excised under the guidance of the preferably real time intra-tissue images of the breast generated by the internal and/or external imaging/interventional device ultrasound transducer(s). Alternatively, the excisional tool may be deployed under both intra-tissue ultrasound as described above and under surface ultrasound, the surface ultrasound being applied to any portion of the stabilized breast that is exposed between the strut(s) of the stabilizer 100, 600, 700 or 800, thereby providing the physician with additional guidance. The tissue sample or lesion may then be biopsied or excised and retrieved, the imaging/interventional device retracted and the incision closed. The method ends at step S16.

According to another embodiment of the present invention, a method of imaging a female breast includes the steps of stabilizing the breast 128 by placing a breast stabilizer 100, 600, 700 or 800 thereon and imaging the breast 128 through a portion thereof that is exposed between the strut(s) 106, 135, 140, 150, 155A or 155B. According to this embodiment, the breast 128 may be imaged through an elastographic or sonoelastic tissue imaging technique. To do this, the stabilized breast may be imaged (via ultrasonic means, for example) when the stabilized breast is in an undisturbed state. The stabilized breast may then be subjected to a mechanical strain, and the strained breast may then be imaged and the images (ultrasound echo data) obtained from the breast in the undisturbed state and strained breast may then be digitally compared. For example, the force applied to the breast by the suction through the through holes 110, 910 may then be varied to generate the mechanical strain. For example, additional suction may be applied to the suction port 108 during the second imaging step to strain the breast or air may be let into the interstitial space(s) 116, again via the suction port 108. This varies the differential pressure between the ambient pressure and the pressure within the interstitial space 116. Other sources of mechanical strain may be used to mechanically strain the breast 128 stabilized in the stabilizer 100, 600, 700 or 800.

By determining and visualizing the local strain levels within the stabilized and strained breast and comparing these strain levels to reference strain levels obtained from imaging the stabilized but undisturbed breast, the elasticity (a measure thereof being its Young's modulus, for example) of the breast tissue may be estimated and visualized as gray-scales of an image such as an elastogram.

While the foregoing detailed description has described preferred embodiments of the present invention, it is to be understood that the above description is illustrative only and not limiting of the disclosed invention. Those of skill in this art will recognize other alternative embodiments and all such embodiments are deemed to fall within the scope of the present invention. Thus, the present invention should be limited only by the claims as set forth below.

What is claimed is:

1. A breast stabilizer for imaging and invasive medical procedures, comprising:
a first annular member adapted to encircle at least a portion of a base of the breast;
a second annular member adapted to encircle at least a portion of an areolar region of the breast, and
at least one strut, the at least one strut mechanically coupling the first annular member to the second annular member.

2. The breast stabilizer of claim 1, wherein the at least one strut includes a facing surface that faces the breast when the stabilizer is in use and wherein an aggregate surface area of the facing surface of the at least one strut is less than 50% of a total surface area of the breast.

3. The breast stabilizer of claim 1, wherein the first annular member defines a first internal lumen and includes a first vacuum port in fluid communication with the first internal lumen and wherein a surface of the first annular member that is adapted to contact the breast defines a plurality of through holes, each of the plurality of through holes being in fluid communication with the first internal lumen.

4. The breast stabilizer of claim 1, wherein the second annular member defines a second internal lumen and includes a second vacuum port in fluid communication with the second internal lumen and wherein a surface of the second annular member that is adapted to contact the breast defines a plurality of through holes, each of the plurality of through holes being in fluid communication with the second internal lumen.

5. The breast stabilizer of claim 1, wherein the at least one strut defines a third internal lumen and includes a third vacuum port in fluid communication with the third internal lumen and wherein the facing surface of the at least one strut defines a plurality of through holes, each of the plurality of through holes being in fluid communication with the third internal lumen.

6. The breast stabilizer of claim 1, wherein the first annular member defines a first internal lumen, the second annular member defines a second internal lumen and the at least one strut defines a third internal lumen, each of the first to third internal lumen being in fluid communication with one another and wherein the stabilizer further includes a suction port in fluid communication with the first to third internal lumen.

7. The breast stabilizer of claim 6, wherein the suction port is disposed on one of the at least one struts.

8. The breast stabilizer of claim 6, wherein the suction port is disposed on one of the first and the second annular members.

9. The breast stabilizer of claim 1, wherein each of the at least one struts is shaped to conform to a shape of the breast.

10. The breast stabilizer of claim 1, wherein three struts mechanically couple the first annular member to the second annular member, each of the three struts being separated from a next adjacent strut by about 120 degrees.

11. The breast stabilizer of claim 1, wherein four struts mechanically couple the first annular member to the second annular member, each of the four struts being separated from a next adjacent strut by about 90 degrees.

12. The breast stabilizer of claim 1, wherein at least one of the first annular member and the second annular member includes a substantially rigid outer layer and a relatively softer inner layer, the softer inner layer, in use, being in contact with the breast.

13. The stabilizer of claim 12, wherein the relatively softer inner layer includes at least one material selected from a group including polyethylene, polyethylene teraphthalate (PET), PETG, PETE and Nylon.

14. A breast stabilizer for imaging and invasive medical procedures, comprising:
a first and a second strut, each including a proximal and a distal end;
a third strut defining a first arc configured to allow at least a portion of a nipple-areolar complex of the breast to protrude therethrough, the third strut being attached to the distal ends of the first and second struts, and
a fourth strut defining a second arc configured to encircle a portion of a base of the breast, the fourth strut being attached to the proximal ends of the first and second struts.

15. The stabilizer of claim 14, wherein a first flange extends from the first strut and a second flange extends from the second strut, the first and second flanges being configured to secure the stabilizer to a flat surface.

16. The stabilizer of claim 14, wherein the stabilizer has a truncated generally semi-conical shape adapted to surround that portion of the breast not resting on the flat surface when the stabilizer is in use.

17. The stabilizer of claim 14, wherein the first to fourth struts each include a substantially rigid outer layer and a relatively softer inner layer, the softer inner layer, in use, being in contact with the breast.

18. The stabilizer of claim 17, further comprising a suction port and wherein at least one of the relatively softer inner layers includes a plurality of through holes in fluid communication with the suction port through an interstitial space between the inner and outer layers.

19. The stabilizer of claim 14, further including a fifth strut including a substantially rigid outer layer and a relatively softer inner layer that is in contact with the breast when the stabilizer is in use, the fifth strut mechanically coupling the first strut to the second strut and/or the third strut to the fourth strut.

20. The stabilizer of claim 19, further comprising a suction port disposed on the fifth strut and wherein the relatively softer inner layer of the fifth strut defines a plurality of through holes in fluid communication with the suction port.

21. The stabilizer of claim 19, wherein at least the third, fourth and fifth struts are integral to one another and each include a substantially rigid outer layer and a relatively softer inner layer that defines a plurality of through holes in fluid communication with the suction port.

22. The stabilizer of claim 18, wherein the first flange defines a plurality of through holes, each of the plurality of through holes being in fluid communication with the suction port, thereby enabling the stabilizer to be secured by suction to the flat surface when suction is applied to the suction port.

23. The stabilizer of claim 14, further including an adhesive layer disposed on a surface of the first and second flanges that faces the flat surface when the stabilizer is in use.

24. The stabilizer of claim 17, wherein the relatively softer layer includes at least one material selected from a group including polyethylene, polyethylene teraphthalate (PET), PETG, PETE and Nylon.

25. The stabilizer of claim 14, wherein the first and second arcs have a generally semicircular shape.

26 The stabilizer of claim 15, further including a third flange extending from the fourth strut, the third flange being oriented to face a chest wall when the stabilizer is in use.

27. The stabilizer of claim 26, wherein the third flange defines a plurality of through holes in fluid communication with the suction port.

28. The stabilizer of claim 18, wherein the suction port includes an elastomeric valve adapted to maintain a pressure differential between ambient pressure and a pressure in the interstitial space between the inner and outer layers.

28. The stabilizer of claim 19, wherein the fifth strut is attached only to the third and fourth struts.

29. The stabilizer of claim 19, wherein the fifth strut is attached only to the first and second struts.

30. The stabilizer of claim 14, wherein the stabilizer has a shape that approximates the shape of an exposed portion of a breast as the breast rests on a substantially flat surface.

31. A method of imaging a female breast, comprising the steps of:
stabilizing the breast by placing a breast stabilizer thereon, the stabilizer including a first annular member adapted to encircle at least a portion of a base of the breast; a second annular member adapted to encircle at least a portion of a peri-areolar region of the breast, and at least one strut, the at least one strut mechanically coupling the first annular member to the second annular member;
imaging the breast through an exposed portion of the stabilized breast.

32. The method of claim 31, wherein the imaging step is carried out using at least one of an ultrasound imaging device and a magnetic resonance imaging (MRI) device.

33. The method of claim 31, wherein the imaging step is carried out using an intra-tissue ultrasound device inserted adjacent the per-areolar region of the stabilized breast.

34. The method of claim 31, wherein the imaging step includes a step of simultaneously disposing two external ultrasound-imaging devices against the stabilized breast.

35. The method of claim 32, wherein the imaging step is carried out through an elastographic tissue imaging technique.

36. The method of claim 35, wherein the elastographic imaging step includes steps of:
carrying out an imaging step on the stabilized breast in an undisturbed state;
subjecting the stabilized breast to a mechanical strain;
imaging the strained breast, and
comparing images obtained from the breast in the undisturbed state and strained breast.

37. The method of claim 36, wherein the stabilizer, in use, attaches to the breast using suction and wherein a force applied to the breast by the suction is varied to generate the mechanical strain.

38. The method of claim 37, wherein the stabilizer includes a suction port and wherein the first and second annular members and the at least one strut include a substantially rigid outer layer and a relatively softer inner layer facing the breast when the stabilizer is in use, the inner layer defining a plurality of through holes in fluid communication with the suction port through an interstitial space between the inner and outer layers and wherein the subjecting step includes a step of varying a differential between ambient pressure and a pressure within the interstitial space.

39. A method of imaging a female breast, comprising the steps of:
stabilizing the breast by placing a breast stabilizer thereon, the stabilizer including a first and a second strut, each of the first and second struts including a proximal and a distal end; a third strut defining a first arc configured to allow at least a portion of a nipple-areolar complex of the breast to protrude therethrough, the third strut being attached to the distal ends of the first and second struts and a fourth strut defining a second arc configured to encircle a portion of a base of the breast, the fourth strut being attached to the proximal ends of the first and second struts, and
imaging the breast through an exposed portion of the stabilized breast.

40. The method of claim 39, wherein the imaging step is carried out using at least one of an external ultrasound imaging device and an MRI device.

41. The method of claim 39, wherein the imaging step is carried out using an intra-tissue ultrasound device inserted adjacent the per-areolar region of the stabilized breast.

42. The method of claim 39, wherein the imaging step includes a step of simultaneously disposing two external ultrasound-imaging devices against the stabilized breast.

43. The method of claim 39, wherein the imaging step is carried out through an elastographic tissue imaging technique.

44. The method of claim 43, wherein the elastographic imaging step includes steps of:
carrying out an imaging step on the stabilized breast in an undisturbed state;
subjecting the stabilized breast to a mechanical strain;
imaging the strained breast, and
comparing images obtained from the breast in the undisturbed state and strained breast.

45. The method of claim 44, wherein the stabilizer, in use, attaches to the breast using suction and wherein a force applied to the breast by the suction is varied to generate the mechanical strain.

46. The method of claim 44, wherein the stabilizer includes a suction port and wherein at least one of the first to fourth struts includes a substantially rigid outer layer and a relatively softer inner layer, the inner layer defining a plurality of through holes in fluid communication with the suction port through an interstitial space between the outer and inner layers and wherein the subjecting step includes a step of varying a differential between ambient pressure and a pressure within the interstitial space.

47. A breast stabilizer for imaging and invasive medical procedures, comprising a cage having an open lattice structure, the cage being shaped to conform to at least a portion of a breast, the cage being adapted to removably adhere to the breast by a selective application of suction, wherein a surface area of the breast at least sufficient to allow access for imaging and/or invasive medical procedures is exposed through the open lattice structure of the cage.

48. The breast stabilizer of claim 47, wherein the stabilizer is re-usable.

49. The breast stabilizer of claim 47, wherein the stabilizer is a one-time use and disposable device.

50. The breast stabilizer of claim 47, wherein the open lattice structure of the cage is configured to expose at least 40% of the surface area of the breast.

51. The breast stabilizer of claim 47, wherein the cage is configured to expose at least a portion of a peri-areolar region of the breast when in use.
